(19) United States
(12) Patent Application Publication
(10) Pub. No.: US 2021/0319850 A1
(43) Pub. Date: Oct. 14, 2021

(54) MICROBIOME-BASED TRACKING SYSTEM AND METHODS RELATING THERETO

(71) Applicant: ADVANCED BIOLOGICAL MARKETING, INC., Geneva, NY (US)

(72) Inventor: Molly Cadle-Davidson, Geneva, NY (US)

(73) Assignee: ADVANCED BIOLOGICAL MARKETING, INC., Geneva, NY (US)

(21) Appl. No.: 17/273,577

(22) PCT Filed: Sep. 6, 2019
(86) PCT No.: PCT/US2019/050055

§ 371 Date: Mar. 4, 2021

Related U.S. Application Data
(60) Provisional application No. 62/728,658, filed on Sep. 7, 2018.

(51) Int. Cl. G16B 30/20; G16B 30/10; C12Q 1/6869

(52) U.S. Cl. CPC G16B 30/20; C12Q 1/6869; G16B 30/10

(57) ABSTRACT

The present invention relates generally to a system and method to identify an origin of one or more products by comparing its microbial composition to known location microbiomes present in a database. The microbiome associated with a single location, such as a farm, should have common elements that differ from all other farms due to a variety of factors including on-farm livestock mix, human inhabitants, soil, water sources, local plant life, climate and weather patterns, local wildlife and native insects, etc. Further inclusion of the microbiome present all along the entire processing and distribution chain will be unique and identifiable due to similar factors as listed above. Methods for metagenomic and microbiome analyses have dramatically improved, making the application of this technology to agricultural product identification and safety a realistic endeavor.

### CROSS-REFERENCE TO RELATED APPLICATIONS

This application is related to U.S. Provisional Application Ser. No. 62/728,658, filed Sep. 7, 2018 entitled “MICROBIOME-BASED TRACKING SYSTEM AND METHODS RELATING THERETO”, which is incorporated by reference herein in its entirety.

### FIELD

The present invention relates generally to microbiome analysis, and more specifically to utilizing metagenomics-based identification and confirmation of the origin of certain products and goods.

### BACKGROUND

The field of metagenomics involves identification of organisms present in a body of water, soil, and other environments of the like. Knowledge of which organisms are present in a particular environment can aid research in ecology, epidemiology, microbiology, and other fields. By sequencing a sample obtained from a certain location, researchers can determine the types of microbes present in that location's microbiome. However, there remains a need in the art for correlating the differences in microbiome profiles of different locations for purposes of tracking the origin of products.

### SUMMARY OF THE INVENTION

The present invention addresses limitations in the art by providing a system for determination of the source or origin of a product, comprising: one or more testable location samples obtained from one or more identified locations, stored in a microbiome reference database; one or more testable product samples obtained from one or more products; one or more sequencers capable of sequencing the one or more testable location samples and the one or more testable product samples to provide sample data from each of the one or more testable location samples in the microbiome reference database and the one or more testable product samples; and a computing device capable of generating a microbiome profile comprising location microbiome data, via a microbiome reference database, and product sample microbiome data that produces the lowest error in sensitivity prediction; wherein the computational algorithm involves (i) a selection of a set of targets that satisfies the identifiable location via the microbiome profile, and (ii) generation of a probabilistic model based on the selected product and its determined location which produces high accuracy sensitivity prediction for product origin with known location microbiome.

In one aspect the testable location sample is obtained from a group consisting of: loading equipment, unloading equipment, handling equipment, personnel, transport interior, transport exterior, facility interior, transport equipment, previous transport load, current and previous load origin, location air samples, processing line equipment, previously processed batch, previous air samples, walls, ventilation systems, soil samples, drinking water, washing water, harvested products, harvesting equipment and tools, crop maintenance equipment and tools, milking machine lines, milk storage, floors, feed, other animals within the location, random sample of livestock, pasture soil/plant life, forage, agricultural crops, and combinations thereof.

In another aspect, the testable product sample is obtained from a group consisting of: food products, agricultural crops, livestock feed, livestock, fiber, textiles, grain, seed, meal, livestock byproducts, oils, botanical extracts, alcohol, water, soil, and combinations thereof.

In another aspect, the sequencing step is selected from the group consisting of: marker gene sequencing, whole metagenome analysis, metatranscriptome analysis, and combinations thereof.

In yet another aspect, the testable location samples are existing location samples in a preexisting networked microbiome reference database capable of query via a network. The testable location sample may comprise previously obtained testable location sample data compiled in a location database, wherein said data further comprises more than one location attributed to more than one products originating from the more than one locations.

In another aspect of the present invention, one or more testable location samples are obtained following identification of one or more products requiring a determination of origin of said one or more products.

It is another object of the present invention to provide a method enabling a computing device to determine the source location of a product comprising: identifying a location; generating a testable location sample from the location; testing viability of the testable sample against one or more sequencing steps; sequencing the testable location sample; identifying a product; generating a testable product sample from the product; testing viability of the testable product sample against one or more sequencing steps; sequencing the testable product sample; generating, via a computing device, a microbiome profile from the sample data that produces the lowest error in sensitivity prediction; wherein the computational algorithm involves (i) a selection of a set of targets that satisfies the identifiable location via the microbiome profile, and (ii) generation of a probabilistic model based on the identified product and its determined location which produces high accuracy sensitivity prediction for product origin with known microbiome profile; and validating the microbiome profile of the testable product sample in vitro against the testable location sample to yield a validated product origin determination.

In one aspect, the sequencing steps are selected from the group consisting of: marker gene sequencing, whole metagenome analysis, metatranscriptome analysis, and combinations thereof.

The testable location sample may further comprise previously obtained testable location sample data compiled in a microbiome reference database, capable of query via a network, wherein said data further comprises more than one location attributed to more than one products originating from the more than one locations.

In another aspect, the one or more testable location samples are obtained following identification of one or more products requiring a determination of origin of said one or more products.

It is another object of the present invention to provide a non-transitory computer readable storage medium configured to store instructions that, when executed by a processor included in a computing device, cause the computing device to confirm the origin of a product, by carrying out steps of: identifying a location; generating a testable location sample from the location; testing viability of the testable sample against one or more sequencing steps; sequencing the testable location sample for populating a microbiome reference database capable of query via a network; identifying a product; generating a testable product sample from the product; testing viability of the testable product sample against one or more sequencing steps; sequencing the testable product sample; generating, via a computing device, a microbiome profile from the testable product sample that produces the lowest error in sensitivity prediction; wherein the computational algorithm involves (i) a selection of a set of targets that satisfies the identifiable location via the microbiome reference database capable of query via a network, and (ii) generation of a probabilistic model based on the identified product and its determined location which produces high accuracy sensitivity prediction for product origin with a known microbiome profile; and validating the microbiome profile of the testable product sample in vitro against the testable location sample to yield a validated product origin determination.

The foregoing summary is illustrative only and is not intended to be in any way limiting. In addition to the illustrative aspects, embodiments, and features described above, further aspects, embodiments, and features will become apparent by reference to the following figures and the detailed description.

### BRIEF DESCRIPTION OF THE DRAWINGS

For a detailed description of the various embodiments, reference will now be made to the accompanying drawings in which:

FIG. 1 illustrates a block diagram of an example computing device that can be configured to implement different aspects of the various techniques described herein, according to some embodiments;

FIG. 2 illustrates an example method, according to some embodiments;

FIG. 3 illustrates a detailed view of a computing device that can represent the computing devices of FIG. 1 used to implement the various techniques described herein, according to some embodiments;

FIG. 4 illustrates an example supply chain, according to some embodiments;

FIG. 5 illustrates a flow diagram for creating a microbiome reference database, according to some embodiments;

FIG. 6 illustrates a flow diagram for a validation step, according to some embodiments; and

FIG. 7 illustrates a flow diagram for a response analysis, according to some embodiments.

FIG. 8 illustrates an example of comparison data relating to multiple forage management practices.

FIG. 9 illustrates an example of comparison data including clustering of samples relating to multiple forage management practices.

### DETAILED DESCRIPTION

The following discussion is directed to various embodiments. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of the embodiment, and not intended to intimate that the scope of the disclosure, including the claims, is limited to that embodiment.

The embodiments described herein set forth techniques for a system—e.g., one or more sensors/detectors, one or more analysis pipelines, and one or more computing devices—to identify an origin of one or more products by comparing its microbial composition to known microbiomes present in a sample. The microbiome associated with a single location, such as a farm, should have common elements that differ from all other farms due to a variety of factors including on-farm livestock mix, human inhabitants, soil, water sources, local plant life, climate and weather patterns, local wildlife and native insects, etc. In fact, a microbiome profile may be specific to a single henhouse (in the case of egg production). Further, the microbiome present all along the entire processing and distribution chain will be unique and identifiable due to similar factors as listed above. Methods for metagenomic and microbiome analyses have dramatically improved, making the application of this technology to agricultural product identification and safety a realistic endeavor.

Following obtaining the microbiome sequence information relating to a location, that information may be stored locally and then transferred to a networked database in some embodiments—a microbiome reference database. The information in the networked microbiome reference database then serves as reference information for further reference to other microbiome data obtained. The microbiome reference database correlates microbiome information, including as a function of time and active management strategies, to a certain location. The microbiome sequence information, including parameters and features thereof may further be organized into an index, a listing, a database, a dictionary, a catalog and so on, referred to as a microbiome reference database capable of query via a network. The result is an ordered set of elements which may include microbiome sequencing data, and the various distinguishing properties or parameters thereof. The identity of the various aspects of the microbiome need not be known. All of those terms describe a list of elements that are included into a single assemblage, wherein the elements are characterized by a plurality of features, wherein any one feature can serve as the basis for ordering the elements in the microbiome reference database.

Microbial communities present in soils and other habitats associated with a particular location, known as the microbiome, provide significant diversity and functional potential relating to carbon cycling pathways and other products and functions having microbial interaction. These functional capabilities have been revealed by the application of high throughput sequencing capabilities, which are capable of identifying the compositions of such microbial communities. These compositions may then be compared and further identified when studied with other microbiomes from other locations. This may be performed without the need for tagging or other modification of a microbiome specific to a location, although these active steps may be incorporated into the claimed invention.

A microbiome profile is thus prepared by collecting testable location samples attributable to a location or supply chain, thereafter sequencing the microbiome of the applicable testable location samples, building a database that associates locations, timepoints, environments, and the like with a product's history/origin, to form the microbiome profile within the microbiome reference database, and interrogating the microbiome reference database with new suspect samples to predict where they came from with a certain probability.

In one embodiment the present disclosure provides a method of profiling a microbiome of a location, comprising: obtaining nucleic acids sequences from greater than one microbe in a biological sample obtained from the location; analyzing said greater than one microbe within said biological sample based upon the nucleic acid sequences obtained; and determining a profile of the microbiome based on said analyzing. In another embodiment, the method can further comprise obtaining nucleic acids sequences of from at least one microbe in a biological sample taken at least two different points of time, or alternatively taken from at least two points within a designated location. In some embodiments, such analyzing uses long read sequencing platforms. Tracking or determination of product provenance can also be accomplished by detection of the various product microbiomes, and therefore no active placement of markers or codes on to produce is required as is previously presented in the art.

High throughput sequencing capabilities have indeed allowed for determination of the diversities of microbiomes across various soil habitats. According to some embodiments, the sequence-based microbiome is capable of establishing a control microbiome fingerprint associated with a location or source of products, termed herein as a microbiome profile. For the purposes of the present disclosure, products may include agricultural crops and forage products, livestock, poultry and poultry products such as eggs, and other food or feed products. Locations may include a single field, henhouse, farm, agricultural region, processing plant, or other locale in the food supply chain, such as transportation and processing. Examples of viable samples within a location include, but are not limited to: air, walls, ventilation systems, random samples, drinking water, washing water, harvested products, and the like. For example, if the location is associated with a hen house, viable samples include but are not limited to: air, walls, ventilation system, random sample from birds, drinking water, washing water, eggs, and the like, as well as combinations thereof. In another exemplary embodiment, if the location is associated with livestock and animals, such as cattle, sheep, pigs, etc., viable samples include, but are not limited to: air, barn walls, bedding, ventilation system, milking machine lines, milk storage, floors, drinking water, washing water, feed, other animals moving through barns, random sample of cattle, pasture soil/plant life, and the like, as well as combinations thereof.

In another exemplary embodiment, if the location is associated with transportation locations or vehicles and storage containers, viable samples include, but are not limited to: loading/handling equipment, personnel, transport interior, transport exterior, transport equipment, previous transport load, current and previous load origin and destination air samples, and the like, as well as combinations thereof. If the location is associated with processing or storage facilities, viable samples include, but are not limited to: unloading/handling equipment, personnel, facility interior, facility exterior, processing line equipment, previously processed batch, current and previous air samples, and the like, as well as combinations thereof. Various products are then capable of being tested to confirm the microbiome characteristics of such product associated with a location or producer. Using comparing logic, the sequence-based microbiome of the product is then correlated to the location of origin.

The microbiomes which are then sampled from the viable samples described herein, are compiled into a microbiome reference database (mrDB) of each domain in a product's life history, the transit, processing and origin of that food product becomes traceable. Associate end product microbiome sample with origin/transport/processing domain with statistical probability. Confidence level is capable of being set to at least 99% confidence. Determination triggers follow up on farm testing for pathogen associated with food/feed-borne illness outbreak.

According to some embodiments, a tracking system can therefore be implemented wherein producers, distribution, and processing facilities all submit samples for input into the microbiome reference database. When a human or livestock food-borne disease outbreak is detected, the contaminated product will be sampled and analyzed for its microbiome profile/composition and compared with the microbiome reference database. The result is the identification with associated probability of the origin of that product and in the best case, all steps in the transit and processing of that product.

The tracking system of the present disclosure can further be used proactively to detect potential for food/feed-borne illnesses prior to distributing the food/feed product to consumers and initiating confirmation and clean up at the indicated point of origin.

According to some embodiments the present disclosure utilizes DNA sequencing and data analysis to determine unique characteristics of a product microbiome of interest, and thereafter comparing samples from products to determine the location, or origin, of the particular product. In one embodiment viable sample or combinations of samples are obtained, which may comprise marker gene sequencing, whole metagenome analysis, metatranscriptome analysis and the like. The system employs a first control sample from one or more known locations to establish baseline control microbiome data. A second sample of a product is then obtained, providing a microbiome profile for said product. By comparing the first control sample data from more than one location, the product can then be confirmed to have originated from the one or more locations, depending on the shipping and processing route environment, determined by the one or more first control samples compared to the second sample relating to the applicable product.

It is noted that the embodiments described herein are primarily directed toward a system for tracking products originating from a location, such as crops, livestock, poultry, equine, mohair/wool, dairy, and products derived therefrom. Unique identifiers are established to allow logic from a controller to determine statistically relevant characteristics when compared to other control samples. Such logic may then be applied to further determine the provenance of the product, including location of origin or authenticity of the product. The farm- or distribution chain-specific microbiome profile can be identified for specific agricultural products and that this “fingerprint” can be used in the tracking of products involved in food-borne illnesses. Not only can this process trace back to the source of human pathogens entering the food chain, but, if implemented early enough in the process, it allows for contaminated goods to be identified prior to reaching a consumer.

Metagenomic approaches to understanding the microbiome of a location stand to help further illuminate the roles of the microbiomes and have only recently been enabled by “next-generation” sequencing technologies. While the information uncovered by such studies will become increasingly valuable to those interested in targeting the microbiome for analysis of products, transforming this large amount of data into meaningful information that can be used to develop quality assurance and confirmation of authenticity presents a significant hurdle. These may be overcome by ensuring that identified characteristics of a microbiome have increased sensitivity and probability. Further descriptions of the methods employed for evaluating microbial communities are set forth in references: (1) Jansson, J., Hofmockel, K. “The Soil Microbiome—from Metagenomics to Metaphenomics” Current Opinion in Microbiology 2018, 43:162-168; and (2) Knight, R. et al., “Best Practices for Analyzing Microbiomes”, Nature Reviews Microbiology, 2018, 16: 410-422, each of which is incorporated by reference in its entirety.

In another embodiment of the present disclosure, samples obtained from identified locations are obtained and analyzed via the disclosed system.

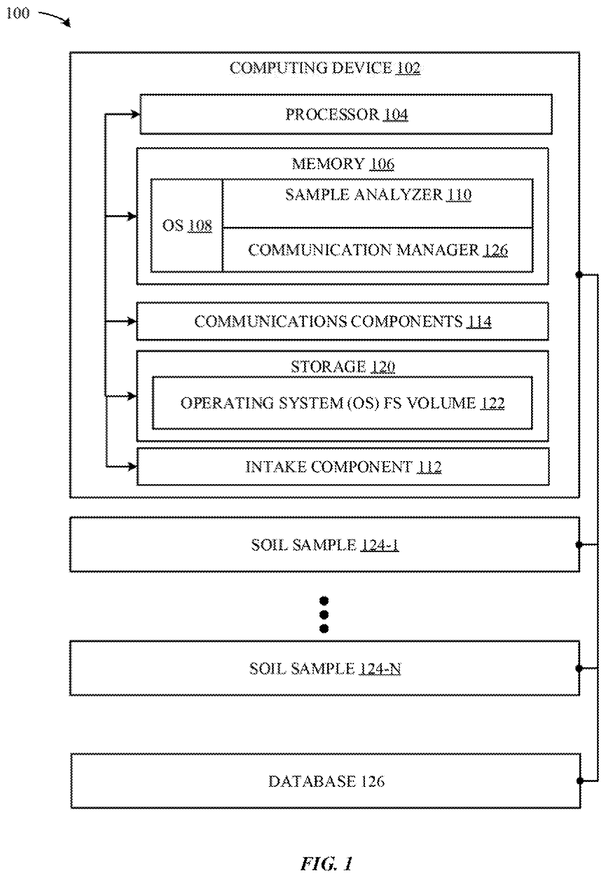

A more detailed discussion of these techniques is set forth below and described in conjunction with FIGS. 1-8, which illustrate example diagrams of systems and methods that can be used to implement these techniques.

FIG. 1 illustrates a block diagram 100 of a computing devices 102 that can be configured to implement various aspects of the techniques described herein, according to some embodiments. Specifically, FIG. 1 illustrates a high-level overview of a computing device 102, which, as shown, can include at least one processor 104, at least one memory 106, and at least one storage 120 (e.g., a hard drive, a solid-state storage drive (SSD), etc.). According to some embodiments, the processor 104 can be configured to work in conjunction with the memory 106 and the storage 120 to enable the computing device 102 to implement the various techniques set forth in this disclosure. According to some embodiments, the storage 120 can represent a storage that is accessible to the computing device 102, e.g., a hard disk drive, a solid-state drive, a mass storage device, a remote storage device, and the like. For example, the storage 120 can be configured to store an operating system (OS) file system volume 122 that can be mounted at the computing device 102, where the OS file system volume 122 includes an OS 108 that is compatible with the computing device 102.

According to some embodiments, and as shown in FIG. 1, the OS 108 can enable a sample analyzer 110 to execute on the computing device 102. It will be understood that the OS 108 can also enable a variety of other processes to execute on the computing device 102, e.g., OS daemons, native OS applications, user applications, and the like. According to some embodiments, the sample analyzer 110 can be configured to analyze the various soil samples 124 to carry out the techniques described herein. According to some embodiments, the sample analyzer 110 can interface with intake component 112 that are included in computing device 102. The intake component 112 can include any type of hardware that is used to sequence one or more samples 124. The sample analyzer 110 may comprise one or more detection elements for obtaining information from a sample, including, but not limited to: marker genes, whole metagenomes and metatranscriptome samples.

Sample analyzer 110 can be configured to further assess and analyze data identified by the intake component 112. For example, the sample analyzer 110 can compare microbiome data associated with a particular sample (soil sample 124-1) with microbiome data stored in a database 126, and so on. It is noted that the foregoing examples are not meant to represent an exhaustive list in any manner, and that any hardware implementing any method that can sequence a soil microbiome can be included in the intake component 112. Further, although the intake component 112 is shown as included in the computing device 102, the intake component 112 can reside in a different computing device that interfaces with computing device 102. Further the data identified by the intake component 112 can be shared with other computing devices where appropriate.

Additionally, and as shown in FIG. 1, the OS 108 can also enable the execution of a communication manager 126. According to some embodiments, the communication manager 126 can interface with different communications components 114 that are included in the computing device 102. The communications components 114 can include, for example, a WiFi interface, a Bluetooth interface, a Near Field Communication (NFC) interface, a cellular interface, an Ethernet interface, and so on. It is noted that these examples are not meant to represent an exhaustive list in any manner, and that any form of communication interface can be included in the communications components 114. In any case, the communication manager 126 can also be configured to interface with the sample analyzer 110 to provide relevant information about soil samples 124. For example—and as described in greater detail herein—the communication manager 126 can receive, via the communications components 114, sequencing data associated with soil samples 124. In turn, the sample analyzer 110 can identify or confirm the source of the soil samples 124 by comparing the sequencing data associated with soil samples 124 to stored data in database 126.

Accordingly, FIG. 1 sets forth a high-level overview of the different components/entities that can be included in computing device 102 to enable the embodiments described herein to be properly implemented. As described in greater detail below, these components/entities can be utilized in a variety of ways to enable the computing device 102 to confirm the origin of source of soil samples 124.

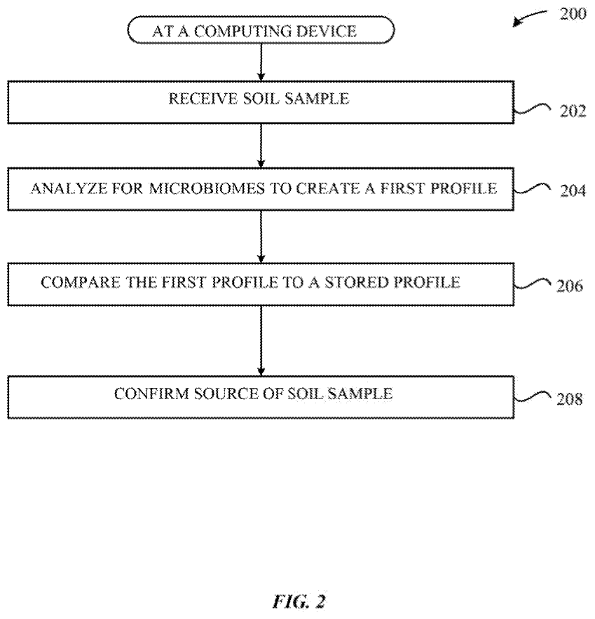

FIG. 2 illustrates a method (200) in accordance with various embodiments disclosed herein. Initially a sample is received at a computing device (202). The computing device analyzes the soil sample for microbiomes to create a first profile (204). Next the computing device compares the first profile to profile stored in a database (206) and confirms the origins or source of the soil sample (208).

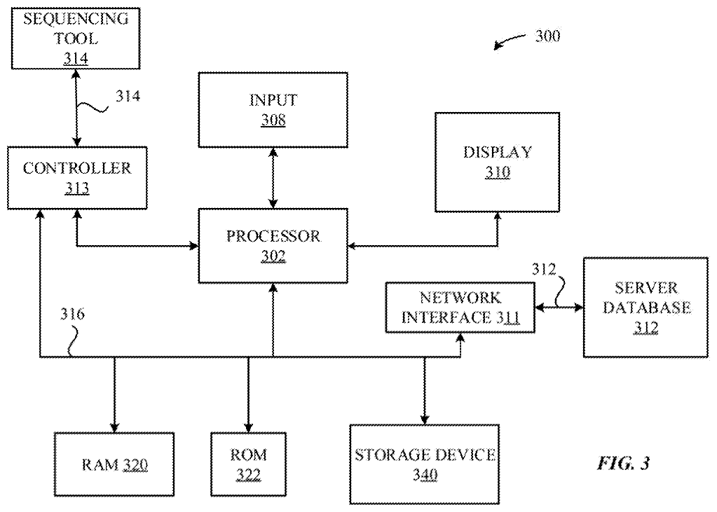

FIG. 3 illustrates a detailed view of a computing device 300 that can represent the computing devices of FIG. 1 used to implement the various techniques described herein, according to some embodiments. For example, the detailed view illustrates various components that can be included in the computing device 102 described in conjunction with FIG. 1. As shown in FIG. 3, the computing device 300 can include a processor 302 that represents a microprocessor or controller for controlling the overall operation of the computing device 300. The computing device 300 can also include a user input device 308 that allows a user of the computing device 300 to interact with the computing device 300. For example, the user input device 308 can take a variety of forms, such as a button, keypad, dial, touch screen, audio input interface, visual/image capture input interface, input in the form of sensor data, and so on. Still further, the computing device 300 can include a display 310 that can be controlled by the processor 302 (e.g., via a graphics component) to display information to the user. A data bus 316 can facilitate data transfer between at least a storage device 340, the processor 302, and a controller 313. The controller 313 can be used to interface with and control different equipment through an equipment control bus 314. For example, the controller 313 can interface with a sequencing tool 314. The computing device 300 can also include a network/bus interface 311 that couples to a data link 312. In the case of a wireless connection, the network/bus interface 311 can include a wireless transceiver.

As noted above, the computing device 300 also includes the storage device 340, which can comprise a single disk or a collection of disks (e.g., hard drives). In some embodiments, storage device 340 can include flash memory, semiconductor (solid state) memory or the like. The computing device 300 can also include a Random-Access Memory (RAM) 320 and a Read-Only Memory (ROM) 322. The ROM 322 can store programs, utilities or processes to be executed in a non-volatile manner. The RAM 320 can provide volatile data storage, and stores instructions related to the operation of applications executing on the computing device 300.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid state drives, and optical data storage devices. The computer readable medium can also be distributed over network-coupled computer systems so that the computer readable code is stored and executed in a distributed fashion.

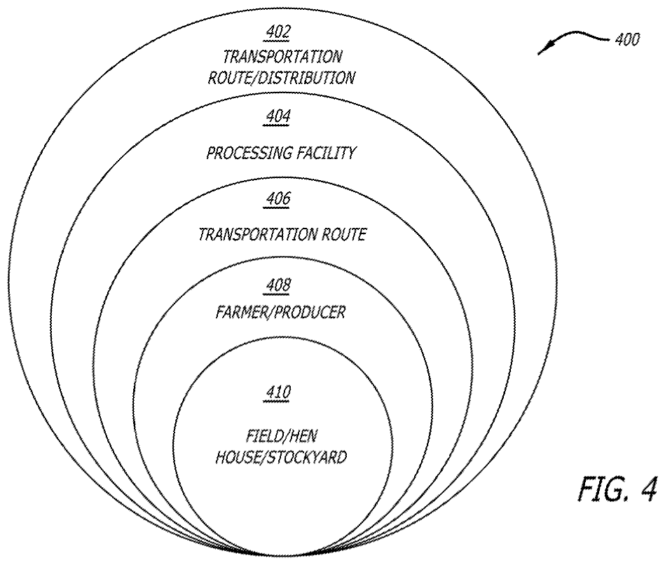

FIG. 4 describes various levels of an exemplary supply chain 400 associated with a food product. Each level in the food supply chain has its own microbiome that is related to the living organisms in the air, water, soil, and other environmental conditions that are routinely present in that domain. Therefore, viable samples may be obtained from the field, hen house or stockyard 410, from the grower or producer 408, from the transportation route 406, from a processing or storage facility 404 as well as from the final distribution channel, including outlets. The various microbiome organisms are discoverable using sampling, sequencing, and computational technology.

One or more example DNA sampling strategies for the field, hen house or stockyard 410 can include samples of the air, walls, ventilation system, a random sample from birds, drinking water, washing water, eggs, and the like. In another example including a domain associated with cattle, a DNA sampling strategy can include samples of air, barn walls, bedding, ventilation system, milking machine lines, milk storage, floors, drinking water, washing water, feed, other animals moving through barns, random sample of cattle, and pasture soil/plant life. In another example including a domain associated with lettuce, a DNA sampling strategy can include samples of air, field soil, random sample of lettuce leaves, maintenance/harvesting equipment, harvesting personnel, farm storage facility/containers, all fields from single farm, farm livestock, non-target farm crops/plants, irrigation water, water ways, and washing water.

An example DNA sample strategy for transportation route 406 can include samples from loading/handling equipment, personnel, transport interior, transport exterior, transport equipment, previous transport load, and current and previous load origin and destination air samples. An example DNA sample strategy for the processing facility 404 can include sample from unloading/handling equipment, personnel, facility interior, facility exterior, processing line equipment, previously processed batch, and current and previous air samples.

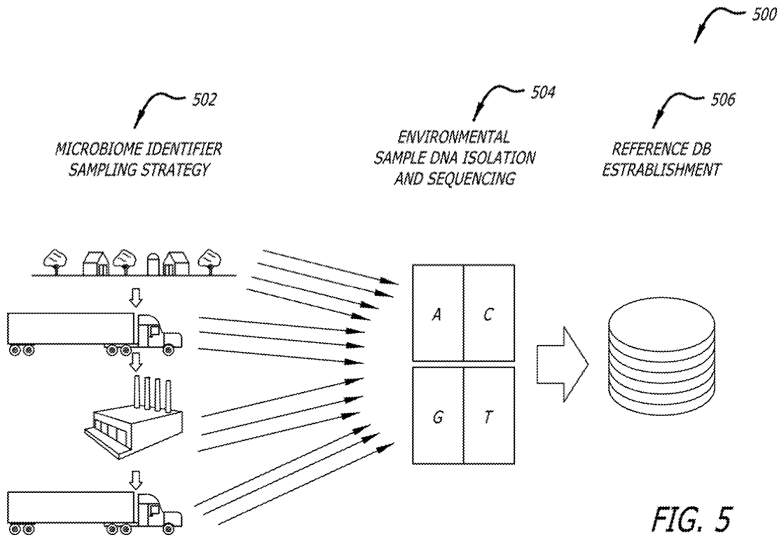

FIG. 5 presents an exemplary description of the location data collection 500 of the present disclosure wherein a microbiome identifier sampling strategy 502 is associated with a location. The testable location sample is subjected to DNA isolation and sequencing 504, where data associated with such DNA isolation and sequencing 504, is then provided to a microbiome reference database 506, wherein the testable location sample from the location, having been analyzed against one or more DNA isolation and sequencing 504 steps.

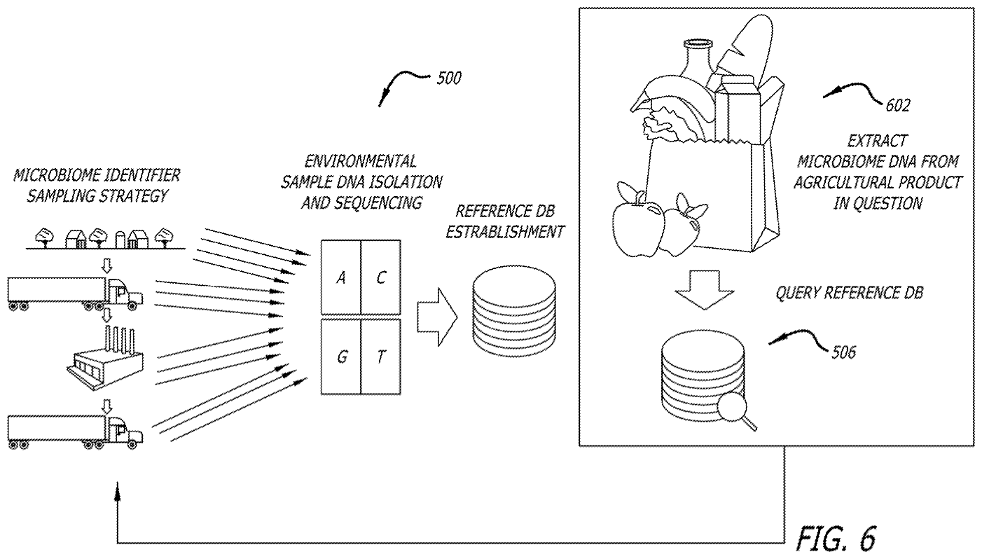

FIG. 6 presents the validation step following development of the microbiome reference database 506. By creating a microbiome reference database 506 of each domain in a food product's life history, the transit, processing and origin of that food product becomes traceable. The process of creating the microbiome reference database 506 can include generating, via a computing device, a microbiome profile from the sample data in the microbiome reference database 506.

After the microbiome reference database 506 is created, a product in question 602 may be traced back through the various domains in the product's life history. This is achieved by extracting microbiome DNA from the product in question 602 and associating the product microbiome sample with origin/transport/processing location with statistical probability producing the lowest error in sensitivity prediction.

One example computational algorithm involves a selection of a set of targets that satisfies the identifiable location via the microbiome profile utilizing the microbiome reference database 506, and subsequent generation of a probabilistic model based on the selected product and its location of origin which produces high accuracy sensitivity prediction for product origin with known microbiome profile. In one embodiment the statistical confidence is at least 95%. In another embodiment the statistical confidence is at least 99%. The system of the present disclosure then validates the microbiome profile in vitro against the testable viable sample to yield a validated product origin determination.

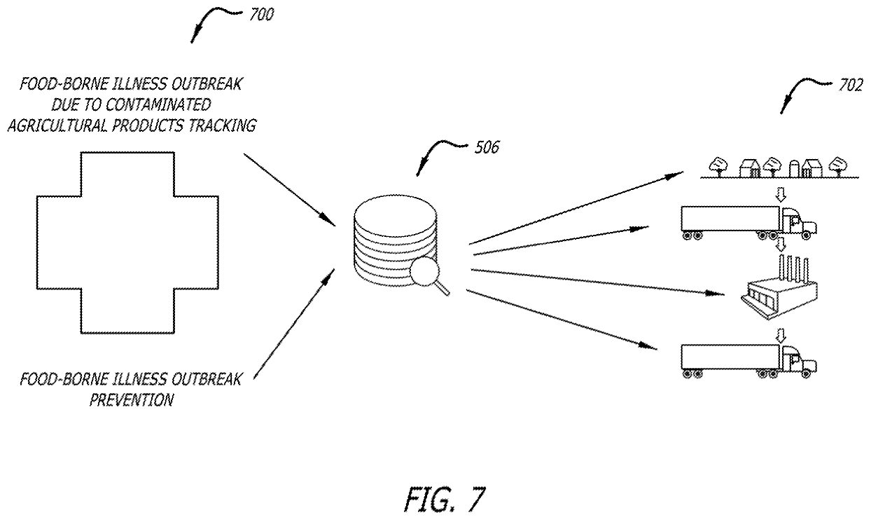

FIG. 7 presents a scenario including a response analysis to an incident, such as a food-borne illness outbreak, including prevention steps. Via the microbiome reference database 506, which contains viable sample data from various locations, the product sample is analyzed by the system, to produce high accuracy sensitivity prediction for the product origin 702 with a known microbiome profile.

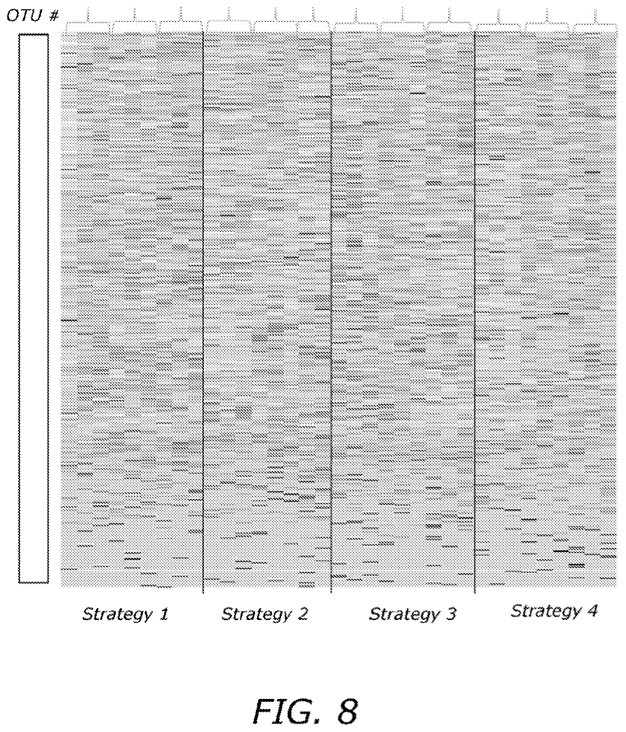

FIG. 8 presents resulting microbiome data from each of three different locations having four differing management strategies applied (see Table 1).

TABLE 1
Management strategies applied to each of three locations.

| No. | Management Strategy |
|---|---|
| 1 | No additional input. |
| 2 | Application of extra Nitrogen. |
| 3 | Application of Trichoderma spp. |
| 4 | Application of Trichoderma spp. + extra Nitrogen |

The data provided in FIG. 8 present a designed microbiome profile showing 121,520 data points relating to a single sequencing experiment. The operational taxonomic units (OTUs) are showing on the vertical axis and represent putative species in the samples. Locations and strategies cluster together and are demarked with brackets along the top axis of the microbiome profile rendering. The active management strategies result in determination of core or defining species for each treatment, allowing for a location or environment to be identified and thereafter added to the microbiome reference database.

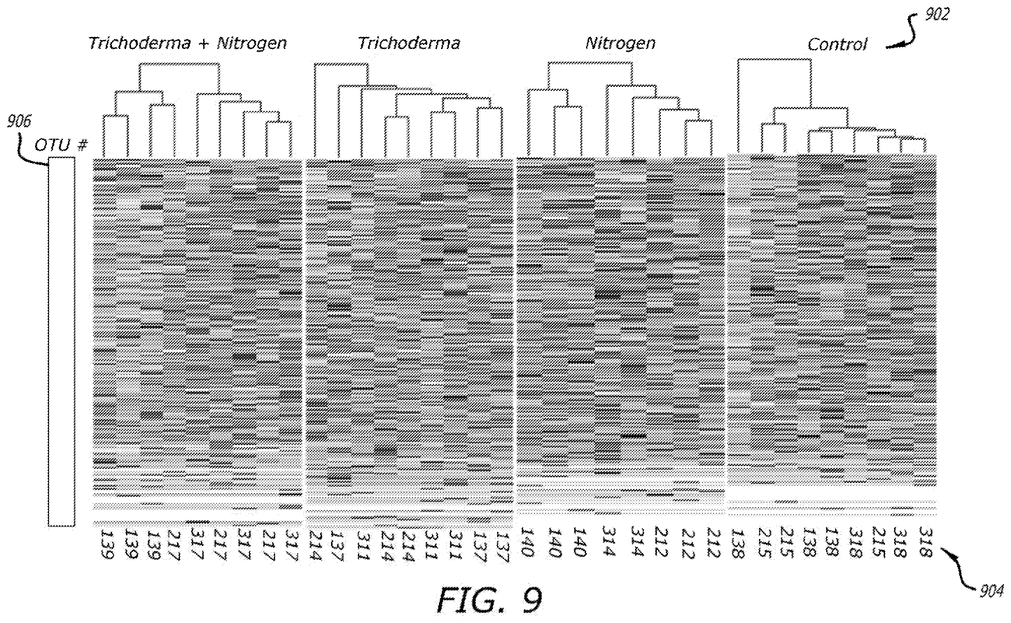

FIG. 9 presents a comparison of microbiome profiles showing forage management practices and the ability to show correlation of different locations associated with good management practices. Clustering of similar samples are provided 902 wherein the x-axis 904 presents location designations. The representations of FIG. 9 show the excellent clustering of samples utilizing common management strategy, with the most significant clustering being associated with good management practices (Nitrogen and Trichoderma+Nitrogen). The microbiome reference database is constructed using all of the microbiome data; however, only those OTUs 906 or species that support more accurate (high confidence) prediction by the algorithm will be used for a given testable location sample.

Thus, a tracking system can therefore be implemented wherein producers, distribution, and processing facilities all submit location samples for input into a reference microbiome database 506, known as the microbiome reference database. When a human or livestock food-borne disease outbreak is detected, the contaminated product will be sampled and analyzed for its microbiome profile/composition and compared with the microbiome reference database 506. The result is the identification with associated probability of the origin of that product and in the best case, all steps in the transit and processing of that product.

The tracking system of the present disclosure can further be used proactively to detect potential for food/feed-borne illnesses prior to distributing the food/feed product to consumers and initiating confirmation and clean up at the indicated point of origin.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

1. A method enabling a computing device to determine the source location of a product comprising:
(a) identifying a location;
(b) generating a testable location sample from the location;
(c) testing viability of the testable sample against one or more sequencing steps;
(d) sequencing the testable location sample;
(e) identifying a product;
(f) generating a testable product sample from the product;
(g) testing viability of the testable product sample against one or more sequencing steps;
(h) sequencing the testable product sample;
(i) generating, via a computing device, a microbiome profile from the sample data that produces the lowest error in sensitivity prediction; wherein the computational algorithm involves (i) a selection of a set of targets that satisfies the identifiable location via the microbiome profile, and (ii) generation of a probabilistic model based on the identified product and its determined location which produces high accuracy sensitivity prediction for product origin with known microbiome profile; and
(j) validating the microbiome profile of the testable product sample in vitro against the testable location sample to yield a validated product origin determination.

2. The method of claim 1, wherein the sequencing steps are selected from the group consisting of: marker gene sequencing, whole metagenome analysis, metatranscriptome analysis, and combinations thereof.

3. The method of claim 1, wherein the testable location sample is obtained from a group consisting of: loading equipment, unloading equipment, handling equipment, personnel, transport interior, transport exterior, facility interior, transport equipment, previous transport load, current and previous load origin, location air samples, processing line equipment, previously processed batch, previous air samples, walls, ventilation systems, soil samples, drinking water, washing water, harvested products, harvesting equipment and tools, crop maintenance equipment and tools, milking machine lines, milk storage, floors, feed, other animals within the location, random sample of livestock, pasture soil/plant life, forage, agricultural crops, and combinations thereof.

4. The method of claim 1, wherein the testable product sample is obtained from a group consisting of: food products, agricultural crops, livestock feed, livestock, fiber, textiles, grain, seed, meal, livestock byproducts, oils, botanical extracts, alcohol, water, soil, and combinations thereof.

5. The method of claim 1, wherein the testable location sample comprises previously obtained testable location sample data compiled in a microbiome reference database, capable of query via a network, wherein said data further comprises more than one location attributed to more than one products originating from the more than one locations.

6. The method of claim 1, wherein one or more testable location samples are obtained following identification of one or more products requiring a determination of origin of said one or more products.

7. A system for determination of the source or origin of a product, comprising:
(a) one or more testable location samples obtained from one or more identified locations, stored in a microbiome reference database;
(b) one or more testable product samples obtained from one or more products;
(c) one or more sequencers capable of sequencing the one or more testable location samples and the one or more testable product samples to provide sample data from each of the one or more testable location samples in the microbiome reference database and the one or more testable product samples; and
(d) a computing device capable of generating a microbiome profile comprising location microbiome data, via a microbiome reference database, and product sample microbiome data that produces the lowest error in sensitivity prediction; wherein the computational algorithm involves (i) a selection of a set of targets that satisfies the identifiable location via the microbiome profile, and (ii) generation of a probabilistic model based on the selected product and its determined location which produces high accuracy sensitivity prediction for product origin with known location microbiome.

8. The system of claim 7, wherein the testable location sample is obtained from a group consisting of: loading equipment, unloading equipment, handling equipment, personnel, transport interior, transport exterior, facility interior, transport equipment, previous transport load, current and previous load origin, location air samples, processing line equipment, previously processed batch, previous air samples, walls, ventilation systems, soil samples, drinking water, washing water, harvested products, harvesting equipment and tools, crop maintenance equipment and tools, milking machine lines, milk storage, floors, feed, other animals within the location, random sample of livestock, pasture soil/plant life, forage, agricultural crops, and combinations thereof.

9. The system of claim 7, wherein the testable product sample is obtained from a group consisting of: food products, agricultural crops, livestock feed, livestock, fiber, textiles, grain, seed, meal, livestock byproducts, oils, botanical extracts, alcohol, water, soil, and combinations thereof.

10. The system of claim 7, wherein the sequencing step is selected from the group consisting of: marker gene sequencing, whole metagenome analysis, metatranscriptome analysis, and combinations thereof.

11. The system of claim 7, wherein the testable location samples are existing location samples in a preexisting networked microbiome reference database capable of query via a network.

12. The system of claim 7, wherein the testable location sample comprises previously obtained testable location sample data compiled in a location database, wherein said data further comprises more than one location attributed to more than one products originating from the more than one locations.

13. The system of claim 7, wherein one or more testable location samples are obtained following identification of one or more products requiring a determination of origin of said one or more products.

14. A non-transitory computer readable storage medium configured to store instructions that, when executed by a processor included in a computing device, cause the computing device to confirm the origin of a product, by carrying out steps as described herein:
(a) identifying a location;
(b) generating a testable location sample from the location;
(c) testing viability of the testable sample against one or more sequencing steps;
(d) sequencing the testable location sample for populating a microbiome reference database capable of query via a network;
(e) identifying a product;
(f) generating a testable product sample from the product;
(g) testing viability of the testable product sample against one or more sequencing steps;
(h) sequencing the testable product sample;
(i) generating, via a computing device, a microbiome profile from the testable product sample that produces the lowest error in sensitivity prediction; wherein the computational algorithm involves (i) a selection of a set of targets that satisfies the identifiable location via the microbiome reference database capable of query via a network, and (ii) generation of a probabilistic model based on the identified product and its determined location which produces high accuracy sensitivity prediction for product origin with a known microbiome profile; and
(j) validating the microbiome profile of the testable product sample in vitro against the testable location sample to yield a validated product origin determination.

15. The non-transitory computer readable storage medium of claim 14, wherein the sequencing steps are selected from the group consisting of: marker gene sequencing, whole metagenome analysis, metatranscriptome analysis, and combinations thereof.

16. The non-transitory computer readable storage medium of claim 14, wherein the testable location samples are existing location samples in a preexisting microbiome reference database capable of query via a network.

17. The non-transitory computer readable storage medium of claim 14, wherein the testable location sample is obtained from a group consisting of: loading equipment, unloading equipment, handling equipment, personnel, transport interior, transport exterior, facility interior, transport equipment, previous transport load, current and previous load origin, location air samples, processing line equipment, previously processed batch, previous air samples, walls, ventilation systems, soil samples, drinking water, washing water, harvested products, harvesting equipment and tools, crop maintenance equipment and tools, milking machine lines, milk storage, floors, feed, other animals within the location, random sample of livestock, pasture soil/plant life, forage, agricultural crops, and combinations thereof.

18. The non-transitory computer readable storage medium of claim 14, wherein the testable product sample is obtained from a group consisting of: food products, agricultural crops, livestock feed, livestock, fiber, textiles, grain, seed, meal, livestock byproducts, oils, botanical extracts, alcohol, water, soil, and combinations thereof.

19. The non-transitory computer readable storage medium of claim 14, wherein the testable location sample comprises previously obtained testable location sample data compiled in a location database, wherein said data further comprises more than one location attributed to more than one products originating from the more than one locations.

20. The non-transitory computer readable storage medium of claim 14, wherein one or more testable location samples are obtained following identification of one or more products requiring a determination of origin of said one or more products.
